(19) United States
(12) Patent Application Publication
(10) Pub. No.: US 2001/0012631 A1
(43) Pub. Date: Aug. 9, 2001

(54) Plant clonal lines and plants having elevated secondary metabolite levels

(75) Inventor: Kalidas Shetty, Amherst, MA (US)

Correspondence Address: RONALD C. LUNDQUIST, PH.D., Fish & Richardson P.C., P.A., Suite 3300, 60 South Sixth Street, Minneapolis, MN, 55402, US

(73) Assignee: University of Massachusetts Medical Center, a Massachusetts Corporation

(21) Appl. No.: 09/750,830

(22) Filed: Dec. 28, 2000

Related U.S. Application Data
(63) Continuation of application No. 08/828,929, filed on Mar. 28, 1997, now abandoned.

(51) Int. Cl. A01H 5/00

(52) U.S. Cl. 435/410; 800/295

(57) ABSTRACT

A method for selecting plants and plant tissue cultures that contain elevated levels of phenolic secondary metabolites is disclosed. The method uses clonal organogenic tissue culture lines, the cultures being derived from meristematic tissue of a member of the Lamiaceae family. Cultured tissue propagules are cultured in the presence of at least one compound that increases the flux through the proline biosynthesis and breakdown pathways. Those clonal lines exhibiting tolerance to the compound have elevated levels of phenolic secondary metabolites. Cultured tissue from such lines can be regenerated into plants, which are used to more efficiently produce essential oils for food and medicinal purposes.

### FIELD OF THE INVENTION

This invention relates to plants that produce elevated levels of phenolic secondary metabolites. More particularly, the invention relates to selection of clonal plant lines of the Lamiaceae family that produce elevated levels of secondary metabolites such as rosmarinic acid.

### BACKGROUND OF THE INVENTION

Plants of the Lamiaceae family are important sources of essential oils for food and medicinal applications, such as agents for treating inflammation, gastrointestinal disorders, and caries. Examples of Lamiaceae species include Hyptis verticillata, Lavandula spp., Orthosiphon aristatus, Rosmarinus officinalis and Mentha spicata, all of which produce rosmarinic acid as a key metabolite. Other examples include Lithospermum erythrorhizon, which produces rosmarinic acid and lithospermic acid as key metabolites. Origanum vulgare produces galangin and rosmarinic acid as key metabolites. Salvia multiorrhiza produces salvianolic acid as a key metabolite. Thymus vulgaris produces thymol as a key metabolite.

Most of the secondary metabolites in essential oils from Lamiaceae plants are phenolic secondary metabolites. For example, rosmarinic acid (α-O-caffeoyl-3,4-dihydroxy-phenyllactic acid) (RA) is one of the secondary metabolites commonly found in Lamiaceae. There is evidence that two aromatic amino acids, phenylalanine and tyrosine, are precursors of phenolic secondary metabolites, including RA, in plants. The proposed pathway for RA biosynthesis is shown in FIG. 1.

Current breeding and selection methods for the family Lamiaceae produce plants with a high degree of plant-to-plant variability in the amount of secondary metabolites present in essential oils from different sources even when extracts are obtained from the same population or geographic production region. The high degree of variability makes it difficult to obtain the metabolite in high quantity and uniformity.

### SUMMARY OF THE INVENTION

The invention is based on the discovery that organogenic plant tissue propagated in culture can be used to select elite clones that produce elevated and uniform levels of phenolic secondary metabolites. These clones are selected based on their tolerance to, e.g., their ability to grow in the presence of, a proline flux-stimulating compound. The growth in culture of clonal lines that produce elevated levels of phenolic secondary metabolites is not inhibited in the presence of such a compound, whereas growth of clonal lines not producing such elevated levels is inhibited in growth. Therefore, there is a correlation between tolerance of the compound and the level of phenolic secondary metabolites in individual clonal lines. Plants regenerated from such selected elite clonal lines can be stimulated to have elevated levels of phenolic secondary metabolites. Because individuals in a clonal population are genetically identical, plants from a selected clonal line have a more uniform level of such secondary metabolites than the corresponding level in individuals of an unselected clonal line.

A method for producing a clonal plant line containing an elevated level of a secondary metabolite is disclosed herein. The method comprises the steps of culturing a first propagule of a Lamiaceae clonal line in the presence of a compound that stimulates proline metabolic flux. The level of the secondary metabolite in the first propagule is compared to the level of the secondary metabolite in a second propagule of the same clonal line which has been cultured in the absence of the compound. The comparison is used to determine whether the propagule has an elevated level of the secondary metabolite relative to the level in the second propagule. If the level in the first propagule is higher, the first propagule is used to produce the clonal plant line. The clonal line can be from the species Mentha spicata, Thymus vulgaris L., Origanum vulgare, Rosmarinus officinalis, Melissa officinalis, or Lavandula augustifolia. The secondary metabolite can be rosmarinic acid, thymol or carvacrol.

The method can further comprise the step of regenerating at least one plant from the first propagule.

The proline-flux stimulating compound can be a competitive inhibitor of proline dehydrogenase. The compound can be, for example, azetidine-2-carboxylic acid.

A Lamiaceae plant is disclosed herein. Such a plant can be produced by culturing a propagule of a Lamiaceae clonal line in the presence of a compound that stimulates proline metabolic flux. A plant is regenerated from the propagule, which has survived culture in the presence of the compound.

The plant can be produced by comparing the level of a secondary metabolite in the cultured propagule to the level of the secondary metabolite in a control propagule of the clonal line cultured in the absence of the compound. The cultured propagule has an elevated level of the secondary metabolite relative to the level in the control propagule. The plant can have an elevated level of the secondary metabolite relative to the level in a plant regenerated from the control propagule.

A method for producing a plant containing an elevated level of a secondary metabolite is disclosed herein. The method comprises the steps of: culturing propagules from a plurality of Lamiaceae clonal lines in the presence of a proline flux-stimulating compound; selecting propagules from at least one line that is tolerant of the compound, and regenerating at least one plant from the selected propagules. The selected propagules contain an elevated level of the secondary metabolite relative to the corresponding secondary metabolite level in control propagules of the selected line cultured in the absence of the compound.

A method for producing an elevated level of a secondary metabolite in a Lamiaceae plant is disclosed herein. The method comprises the steps of: obtaining a Lamiaceae plant selected for tolerance to a proline flux-stimulating compound; contacting the plant with the compound; and growing the plant for a time sufficient to produce an elevated level of the secondary metabolite. The compound can be, for example, hydroxyproline.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

The present invention advantageously results in a more uniform phenolic secondary metabolite content in Lamiaceae plants, thus providing more predictable metabolite production in commercial fields. The present invention also decreases the cost of producing medicinal and food products from Lamiaceae plants.

### BRIEF DESCRIPTION OF THE DRAWINGS

FIG. 1 is a diagram showing the proposed biochemical pathway from phenylalanine and tyrosine to rosmarinic acid (RA).

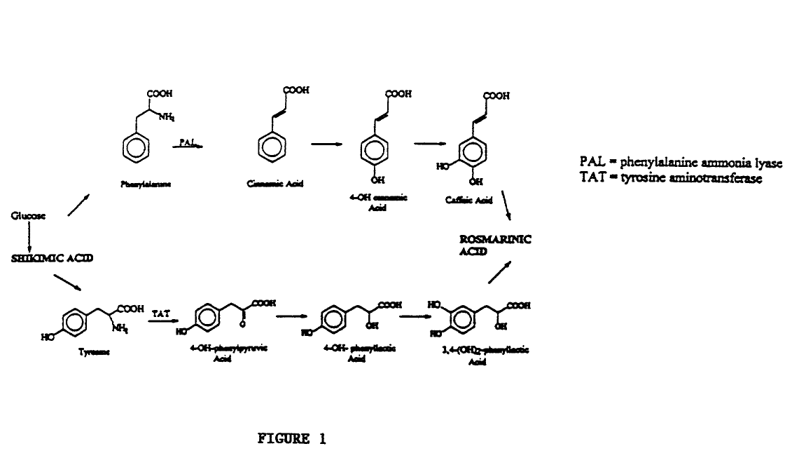

FIG. 2 is a diagram showing the postulated link between proline metabolism, the pentose phosphate pathway and RA biosynthesis.

### DETAILED DESCRIPTION

The presence of genetic heterogeneity in populations of thyme, sage, oregano, rosemary and other plants of the family Lamiaceae results in high variability in phenolic secondary metabolite content in individual plants. This heterogeneity poses problems for predictable commercial production of secondary metabolites from populations of such plants and increases the cost of producing medicinal and food products from such herb and spice plants.

The rationale for the use of proline flux-stimulating compounds in the new methods is based on a hypothesis, proposed herein, concerning the relationship between the pentose phosphate pathway and proline metabolism. The pentose phosphate pathway is an alternate route for the breakdown of carbohydrates. Important functions of this pathway are to generate NADPH for use in biosynthetic (anabolic) reactions and to provide ribose-5-phosphate for nucleotide biosynthesis and erythrose-4-phosphate for the shikimate pathway.

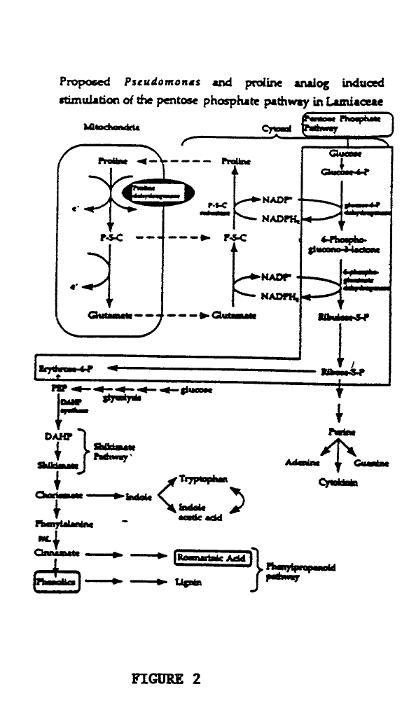

According to the hypothesis, a pentose phosphate pathway exists in plants that is linked to proline biosynthesis via an NADPH/NADP redox cycle. Increased rates of proline synthesis in this pathway result in an increased rate of synthesis of erythrose-4-phosphate (E-4-P). See FIG. 2. An increased rate of synthesis of E-4-P is proposed to result in an increased rate of synthesis of secondary metabolite precursors in the shikimate and phenylpropanoid pathways, such as phenylalanine and tyrosine. Increased levels of such precursors results in increased levels of phenolic secondary-metabolites such as rosmarinic acid.

A clonal line capable of growth in the presence of a proline flux-stimulating compound has, via the proposed proline-linked pentose phosphate pathway, an increased level of E-4-P. E-4-P is directed into the shikimate and phenylpropanoid pathways and results in increased levels of phenolic secondary metabolites, end products of the shikimate and phenylpropanoid pathways. Pseudomonas-tolerant clonal lines have been round to also be tolerant of proline flux-stimulating compounds and have elevated levels of phenolic secondary metabolites.

In the new methods, populations of organogenic plant tissue are generated from clonal lines. Each population originates from a single heterozygous plant or plant part or “organ.” Subsequently, tissue of each clonal population is cultured in vitro on media supplemented with a proline flux-stimulating compound. Those clonal lines that grow in the presence of the compound are more likely to have high levels of phenolic secondary metabolites, such as rosmarinic acid, thymol or carvacrol, than unselected clonal lines.

Clonal lines are generated from a species in the Lamiaceae family. Such species include, without limitation, Mentha piperita, M. arvensis, M. spicata, M. viridis, Borage officinalis, Melissa officinalis, Ocimum gratissimum, O. sanctum, O. basilicum, Salvia officinalis, Thymus vulgaris L., Origanum vulgare, Lavandula augustifolia and Rosmarinus officinalis.

Clonal lines are generated from organogenic cultures of a desired plant species. Such cultures typically are generated from seeds, axillary buds, shoot meristems, or cut cotyledons of the desired plant species. A preferred organogenic culture is a shoot organogenic tissue culture. Alternatively, embryogenic callus cultures may be used, generated from the same plant tissues as those used to generate organogenic cultures. Clonal lines can be generated, multiplied, and maintained indefinitely in tissue culture by subculturing. Preferably, a plurality of organogenic lines are generated, each line derived from a different plant. Each line preferably is multiplied in culture so that a plurality of tissue propagules from each line are available for subsequent steps. Since each population is derived from a single genotype, individual tissue propagules within each population are genetically identical.

Organogenic cultures typically are initiated on semi-solid media containing plant hormones appropriate to generate the desired type of organ. Such media typically comprise salts, vitamins, an energy source, an osmotic agent, a gelling agent, and the like. For example, shoot organogenic tissue culture may use a Murashige and Skoog (MS) media formulation comprising 3% sucrose, Gelrite™ and a cytokinin such as benzyladenine, kinetin, thidiazuron, zeatin, or adenine sulfate. For some species, media is also supplemented with an auxin, to provide an appropriate balance of hormones that promotes tissue growth and organ generation.

An example of a media formulation for oregano is: 4.3 g/l MS salts, 10 ml/l Nitsch and Nitsch basal salt mixture (Sigma), 20 g/l sucrose, 2.75 g/l Phytagel™, and 1 mg/l benzylaminopurine. Media formulations suitable for initiating and maintaining an organogenic culture are developed by techniques known to the skilled artisan, e.g., testing various amounts of salts and hormones to identify an appropriate and optimum concentration for each component.

Clonal lines of oregano and holy basil can be initiated and propagated via axillary shoot proliferation. Shoot organogenesis is induced by culturing seeds on MS media supplemented with benzyladenine. Individual shoots are generated along the nodes of proliferating apex explants. About 5-10 clonal shoots are induced per explant. Clonal lines are maintained and expanded by subculturing meristematic tissue every 20-60 days.

In rosemary and thyme, adventitious bud proliferation can be used to generate multiple shoots from explants originating from a single heterozygous seed. Multiple shoots originate from a single node on each explant and are cultured on MS medium with a cytokinin such as benzylaminopurine. Individual shoots are excised and transferred to the same medium to propagate and multiply each clonal line.

The new method comprises incubating cultured tissue propagules of at least one clonal line on media supplemented with at least one compound that stimulates metabolic flux through the proline biosynthesis and breakdown pathways. Suitable proline flux-stimulating compounds include competitive inhibitors of proline dehydrogenase. One suitable compound is azetidine-2-carboxylic acid (A2C). Other suitable proline-flux stimulating compounds are thiaproline (L-thiazolidine-4-carboxylic acid), and 5-oxo-proline (L-5-oxo-2-pyrrolidine-carboxylic acid, pyroglutamic acid), commercially available from Sigma Chemical Co., St. Louis, Mo. Five-oxo-proline can also be prepared by autoclaving a solution of glutamine and isolating 5-oxo-proline therefrom. Another proline flux-stimulating compound is hydroxyproline. Physiologically acceptable salts and other forms of the proline-flux stimulating compound are also suitable.

The concentration of the proline flux-stimulating compound or compounds in the selection medium depends upon the tissue and species under selection and the particular compound being used. A suitable concentration sufficiently inhibits lines that do not produce elevated levels of phenolic secondary metabolites, while allowing relatively uninhibited growth of lines that achieve elevated levels of such metabolites.

A suitable concentration of A2C for most Lamiaceae species is from about 50 μM to about 350 μM. For example, a suitable concentration of A2C for selection of oregano shoot tissue is from about 50 μM to about 350 μM, e.g., about 75 μM to about 300 μM, or about 100 μM to about 200 μM. A suitable concentration of 5-oxo-proline for most Lamiaceae species is from about 100 μm to about 1 mM, e.g., about 200 μM to about 800 μM, or about 400 μM to about 600 μM. A suitable concentration of thiaproline or hydroxyproline for most Lamiaceae species is from about 500 μM to about 3 mM, e.g., about 750 μM to about 3 mM, or about 1 mM to about 3 mM.

A plurality of cultured tissue propagules of a clonal line are cultured in the presence of the proline flux-stimulating compound. Tissue propagules are cultured on selection media that supports growth and proliferation of new organs, e.g., shoots. Alternatively, propagules can be cultured on selection media that supports regeneration of plants, discussed hereinbelow. Incubation conditions are adjusted to take into account the tissue and species being incubated. For example, conditions for optimum growth of thyme shoot organogenic tissue is about 23° C. and 16/8 hours day/night light cycle at a light intensity of about 2000-3000 lux.

Tissue propagules are incubated for a sufficient period of time in a growth room or growth chamber to allow selection to occur, typically from about 10 to about 60 days. The length of the incubation period depends upon the growth rate of the particular tissue and species under selection. For example, an incubation period of about 10 to about 60 days, e.g., from about 15 to about 30 days, or about 20 to about 30 days is used for thyme shoot tissue. For oregano shoot tissue, an incubation period of about 10 to about 60 days, e.g., from about 20 days to about 40 days, or from about 30 days to about 40 days is used.

The proportion of lines that grow and survive in the presence of a proline flux-stimulating compound depends upon the species under selection and the concentration of the compound. Typically, from about 0.1% to about 20% of lines exposed to selection will grow and survive. Most of the selected lines produce elevated levels of phenolic secondary metabolite. Not all lines produce elevated levels of phenolic secondary metabolites, probably because there are biochemical mechanisms for resistance to the proline-flux stimulating compound other than the mechanism that elevates phenolic secondary metabolites. For example, a membrane permeability mutation could prevent uptake of the compound. Such a line would be resistant yet not have high levels of phenolic secondary metabolites.

During the incubation period, tissue propagules of those clones that are genetically predisposed to tolerate the presence of the compound will grow and survive. Such clones are not significantly inhibited by the presence of the compound. Tissue propagules of such clones produce elevated concentrations of phenolic secondary metabolites.

Tissue propagules of clones that cannot tolerate the presence of the inhibitor will become necrotic and die. Such lines exhibit symptoms of hyperhydricity or vitrification, which is a physiological malformation affecting clonally propagated plants in tissue culture. Such malformed tissues are enlarged, thick, translucent, and brittle. This phenomenon is associated with chlorophyll deficiency, poor lignification, and excessive hydration of tissues.

As a control, parallel incubation is carried out using a plurality of tissue propagules from each clonal line, of like age and subculture regimen, that are not incubated in the presence of the compound. Such a control incubation allows more accurate quantitation of the increase in secondary metabolite concentration for compound-tolerant clones. Such a control incubation also ensures that the necrosis observed in compound-sensitive clones is due to the compound rather than some other abiotic or biotic factor.

At the end of the incubation period, tissue propagules from clones tolerant to the compound are analyzed for the concentration of at least one phenolic secondary metabolite. Such secondary metabolites include, without limitation, rosmarinic acid, thymol, carvacrol, salvianolic acid, lithospermic acid, and various flavonoids. If desired, a plurality of phenolic secondary metabolites can be determined, e.g., the carvacrol, thymol, and rosmarinic acid content can be measured in oregano or thyme. The particular metabolite or metabolites chosen to be measured will, of course, depend upon the species and cultivar under selection. It is known that each species and cultivar has a particular spectrum of secondary metabolites that can be produced by that species and cultivar.

Secondary metabolite levels are measured by gas-liquid chromatography (GC), gas chromatography/mass spectrometry (GC/MS), high performance liquid chromatography (HPLC) or spectrophotometry. If desired, the total phenolic content can be estimated, e.g., by using Folin-Ciocalteu reagent to measure hydroxylated phenolic secondary metabolites and flavonoids. Such a measurement is often simpler and less expensive for initial screening of a plurality of clonal populations, although not necessarily as accurate for a particular secondary metabolite as GC, GC/MS, HPLC or spectrophotometry. The total phenolic content of selected lines is higher, on a fresh weight basis, than the content of compound-sensitive lines. The total phenolic content is also often higher on a dry weight basis.

Total phenolic content can be measured, for example, using Folin-Ciocalteu reagent essentially as described in Chandler and Dodds, Plant Cell Rep. 2:105-108 (1983) and Shetty, K., Ph.D., Thesis, University of Idaho (1989). The tissue is disrupted in 95% ethanol and centrifuged to remove particulate matter. An aliquot of the ethanol supernatant is mixed with an equal volume of Folin-Ciocalteu reagent, and incubated at 25° C. A 5% sodium carbonate can be added to stabilize color development. Absorbance of the solution is measured at 725 nm is a spectrophotometer. A standard curve is developed using various concentrations of gallic acid in 95% ethanol. Absorbance values are converted to mg total phenolics/g fresh weight (FW) tissue from the standard curve.

Secondary metabolite content can be measured by gas chromatography with flame ionization. Tissue is extracted with n-pentane by steam distillation, and extracts are analyzed with gas chromatograph. Peak assignments can be confirmed by GC/MS.

The secondary metabolite concentration in tissue exposed to at least one proline flux-stimulating compound is also compared to the corresponding concentration in tissue from the same clonal line that has been cultured under similar conditions but in the absence of exposure to the compound. Those lines in which the secondary metabolite concentration is higher in the tissue exposed to the proline flux-stimulating compound, on a fresh weight, or dry weight basis, are considered to be elite lines.

The total phenolic content and/or secondary metabolite concentration in propagules from elite lines is higher than the corresponding level in control propagules by about 2-fold to about 4-fold; in some cases the increase is up to about 8-fold.

For some species, it is known that secondary metabolites accumulate in special organs in the plant. For such species, it may be desirable to determine the secondary metabolite level in, or to extract the metabolites from, plants regenerated from tissue propagules surviving the compound selection process, after such plants have developed the appropriate organ. For example, thymol analysis typically is done at later developmental stages when leaf glandular cells for thymol accumulation are more fully developed. Analyses of the whole plant can be carried out in addition to analyses of tissue propagules, if desired.

After selection and identification of clonal lines producing elevated levels of at least one secondary metabolite, tissue propagules of selected lines can be regenerated into plants. Regeneration is accomplished by means known in the art. Typically, tissue propagules are transferred to semi-solid media containing reduced levels of plant hormones. For example, oregano shoot tissue is transferred to half-strength Murashige and Skoog (MS) medium that contains no hormones. An example of a suitable medium is 2.17 g/l MS salts, 5 ml of Nitsch and Nitsch basal salt mixture (Sigma), 15 g/l sucrose, and 2.75 g/l Phytagel™ at pH 5.8. After plantlets have formed, they are hardened off, transferred to potting soil, and allowed to mature, and set seed.

Propagules can be transferred to a regeneration medium immediately after an initial cycle of selection. More than one cycle of selection in culture can be performed before transferring to regeneration medium. Alternatively, and preferably, selection and regeneration can be carried out concomitantly on propagules cultured in regeneration medium.

Tissue-culture based propagation provides a means for generating clonal lines, each line originating from a single seed among a heterogeneous and heterozygous population of seeds. The new method can be applied to any species that is open-pollinated and, therefore, comprises genetically heterogeneous populations. Interestingly, clonal lines identified as tolerant of a proline flux-stimulating compound often exhibit delayed subculture cycles and senescence after subsequent culturing in the absence of the proline flux-stimulating compound.

The use of organogenic tissues in culture to generate clonal lines is advantageous in that organogenic cultures do not require extensive and complicated hormone combinations for propagation. The direct use of organogenic cultures also avoids the use of an intermediate callus stage when selecting desired plant clones and the potential problems associated with callus cultures, such as genetic instability.

A plant of a clonal line according to the invention contains an elevated level of at least one phenolic secondary metabolite. A population of plants from the same clonal line contain such a metabolite at an elevated and more uniform level than that of an unselected population. This is so because all of the individuals in the selected population are genetically identical. Although each plant in the population is heterozygous, substantially all of the plants have the same genotype and therefore respond in a similar manner to growth and environmental conditions by producing a similar concentration of secondary metabolites. Such plants can be propagated indefinitely from vegetative cuttings. Because the population is heterozygous, plant populations from subsequent, sexually-produced generations will not necessarily produce uniform levels of secondary metabolites.

A proline flux-stimulating compound can be used to stimulate or maintain elevated phenolic secondary metabolite levels in plants derived from elite lines in a greenhouse or outdoor environment. Plants from elite lines in such an environment can be exposed to such a compound by, e.g., spraying a solution of the compound on a production field prior to harvest using sprayer means known in the art. Each plant typically receives about 1 to about 5 ml of solution on the upper surface of the leaves.

A solution to be sprayed includes a proline flux-stimulating compound and optionally includes a surfactant to promote entry of the compound into plant cells, and a stabilizer to increase shelf life. If A2C is chosen as the proline-flux stimulating compound, it is present in the solution at a concentration from about 20 μM to about 500 μM, e.g., from about 40 μM to about 250 μM, or from about 50 μM to about 150 μM. For economic reasons, a preferred compound for use on plants is hydroxyproline. Hydroxyproline is applied at about 0.2 mM to about 10 mM, e.g., about 0.4 mM to about 5 mM, or about 0.5 mM to about 1 mM. An inexpensive source of hydroxyproline is soluble fish protein hydrolysate (SFPH), made from the solid waste left behind after fish processing (Mackie, Process Biochem., 17:23-31 (1982). Ionic and nonionic surfactants suitable for use in the invention are known in the art and include, without limitation, Nonidet P40™, Triton X102™ and Brij 35™.

Alternatively, a proline flux-stimulating compound can be applied to the plant root system, e.g., with agricultural equipment designed to apply insecticides or other chemicals. A compound preferably is applied side band rather than in-furrow. The time before harvest at which plants are exposed to the compound depends upon the species and environmental factors, but typically is about 2 to about 14 days before harvest, e.g., about 2 to about 10 days, or about 2 days to about 7 days. The proline flux-stimulating compound can be applied as pellets, granules or in solution. If the compound is applied in solution; each plant receives from about 5 ml to about 75 ml, e.g., from about 10 ml to about 40 ml, or from about 15 to about 20 ml, at concentrations similar to those used for foliar application.

In some embodiments, a solution applied to plants comprises extracellular mucoid component (EMC) in addition to a proline flux-stimulating compound. EMC includes mucopolysaccharides, e.g., galactoglucans, glycoamino-glucans, and other polysaccharides that are highly viscous in concentrated form. EMC is produced by bacterial species such as Pseudomonas spp. or by saprophytic fungi such as Trichoderma pseudokoningii or T. harzianum. EMC is included in the solution at from about 0.1% to about 5% weight/volume, e.g., about 0.2% to about 2%, or about 0.4% to about 1%. EMC can be prepared as described in U.S. application Ser. No. 08/771,241, filed Dec. 20, 1996, incorporated herein by reference. EMC provides useful physical properties to the solution such that the solution covers a larger surface area than would otherwise be the case. EMC may also act as a mild elicitor.

### EXAMPLES

The invention will be further described in the following examples, which do not limit the scope of the invention described in the claims.

### Example 1

### Proline and RA Content in Pseudomonas Tolerant and Inhibited Clonal Lines of Oregano

A direct shoot organogenesis system was developed without an intermediate callus stage for oregano. Clonal lines of oregano were generated from individual seedlings following germination of a heterogeneous seed population (C.S. Hart Co., Chesterfield, Conn.). About 200 seeds were disinfected by immersing for 90 seconds in 70% ethanol and 20 minutes in 2% sodium hypochlorite. The seeds were then rinsed 3 times with autoclaved water for 5 minutes each and transferred to water-agar plates with 0.8% agar (Sigma Chemical Co., St. Louis, Mo.). After 30 days of germination, individual shoot apices arising from multiple areas of each seedling were excised aseptically and transferred to petri plates containing Murashige and Skoog (MS) salts, Nitsch and Nitsch basal salts, 1 mg/l benzylaminopurine (BAP), 5 mM proline, 3% sucrose, and a gelling agent. All tissue culture media and chemicals were purchased from Sigma Chemical Co., St. Louis; Mo. The initial medium pH was 5.8. Each petri plate had 8 apices with each apex having two lateral leaves below it.

Petri plates containing shoot apex explants were incubated at 24° C., for a 16 hour light cycle with a light intensity of 40 μmol.m⁻².s⁻¹. About 70% of the seeds were lost due to seed-borne infection. After 30 days, uncontaminated shoot-apex explants had regenerated several more shoot apices through axillary shoot proliferation.

About 7-10 new shoot apices typically were obtained from each original explant. Additional shoots were obtained from each clonal line by subculturing the original shoot explants at 30-day intervals on MS medium containing BAP. Shoots could be subcultured indefinitely to generate more adventitious shoots. About 100 clonal lines were developed, each clonal line originating from a different heterozygous seed. Lines 0-5, OM-8, 0-1, OM-1 and 0-4 were used in the experiments described herein.

A mucoid, non-pathogenic Pseudomonas sp. strain was isolated as a contaminant of an oregano shoot culture. This Pseudomonas sp. was termed strain F. This strain was used in selecting Pseudomonas tolerant clonal lines. Strain F was grown on yeast extract-mannitol medium (Difco, Inc., Detroit, Mich.) until the inoculum density was about 10⁹ colony forming units/mL. The bacterial suspension was diluted 100-fold in sterile distilled water and about 2.5 ml was dispensed into petri plates. Individual shoots of each clonal line at about 30 days of subculture were removed by cutting at the basal end and then inoculated by dipping the cut end into the diluted bacterial suspension. Inoculated shoots were then transferred to half-strength hormone-free MS medium, which induces root formation, and incubated in a growth room at 23° C., on a 16/8 hours day/night schedule. About 40 shoots of each clone were inoculated. Control shoots of each clonal line were transferred to the same medium, but were not exposed to bacteria.

Samples of Pseudomonas-treated and untreated control shoots from each line were assayed for rosmarinic acid content and proline content. Rosmarinic acid was extracted from 50 mg of fresh shoot tissue using 2 ml of 50% (v/v) methanol and incubating for 1 hour at 55° C. One ml of the methanol extract was diluted 1:18 and the absorbance of the diluted extract was measured at 333 nm in a Genesys spectrophotometer (Spectronic Instruments, Rochester, N.Y.). The concentration of RA in the extract was calculated using an extinction coefficient of ε₃₃₃=19,000 τmol⁻¹cm⁻¹. Values for the concentration of RA for each treatment/clonal line shown in the Tables and Figures of this application are the average of at least 5 separate extracts. Total rosmarinic acid content was expressed as % g/g fresh weight (FW) of tissue.

Proline content was determined essentially as described by Bates et al., Plant and Soil 39:205-207 (1973). Briefly, about 0.5 gm of fresh tissue was homogenized in 10 ml of 3% aqueous sulfosalicylic acid and the homogenate was filtered through Whatman® #2 paper. Two ml of the filtrate were incubated with 2 ml of acid-ninhydrin (ninhydrin/acetic acid/phosphoric acid) and 2 ml of glacial acetic acid for 1 hour at 100° C. The reaction was terminated by cooling the tube in an ice bath. The mixture was shaken vigorously with 4 ml of toluene and the toluene phase removed. The absorbance of the toluene phase was read at 250 nm, using toluene as a blank. Proline concentration was determined from a standard curve and the amount on a fresh weight basis was calculated as: [(μg proline/ml×ml toluene)/115.5 μg/μmole]/[(gm sample)/5]=μmole proline per gm fresh weight sample.

Pseudomonas tolerance of Pseudomonas-treated shoots was determined by visual observation with a stereomicroscope (Olympus-SZ40, Tokyo, Japan) at a magnification of 34X. Cultured shoots were evaluated for morphological abnormalities, including malformation, necrosis, and chlorophyll deficiency. The response of individual tissue propagules to Pseudomonas contact was similar within each line, i.e., there was little or no variation among tissue propagules within each line.

Results for two clonal shoot organogenic lines are shown in Table 2. Clonal line 0-4, which is able to grow after exposure to Pseudomonas strain F, has increased levels of proline and RA at 30 and 41 days after inoculation, compared to the untreated control. Clonal line 0-5, which is inhibited in growth after exposure to strain F, does not have increased levels of proline or RA at 30 days after inoculation, compared to the untreated control.

TABLE 2
Rosmarinic Acid and Proline Levels of
Oregano Clonal Lines 0-4 and 0-5 after
Exposure to Pseudomonas sp.

| Clonal | Pseudomonas | RA | Proline |
|---|---|---|---|
| Line | Treatment | (mg/g FW ± SD) | (μmol/g FW ± SD) |
| 0-4^{a} | Control | 1.7 ± 0.1 | 21.25 ± 7.3 |
| | Inoculated | 3.9 ± 0.5 | 36.37 ± 6.6 |
| 0-5^{a} | Control | 2.8 ± 1.3 | 14.82 ± 4.7 |
| | Inoculated | 2.2 ± 0.7 | 14.93 ± 9.2 |
| 0-4^{b} | Control | 3.3 ± 0.6 | 9.34 ± 0.8 |
| | Inoculated | 7.1 ± 1.4 | 26.64 ± 4.2 |

^{a}Analyzed 30 days after exposure to Pseudomonas.
^{b}Analyzed 41 days after exposure to Pseudomonas.

### Example 2

### Proline Analog Tolerance of High and Low RA-Producing Clonal Lines of Oregano

Twenty clonal oregano lines exhibiting tolerance to strain F and twenty lines exhibiting inhibition in the presence of strain F were tested for cross-tolerance to azetidine-2-carboxylate (A2C). Individual shoot tissue propagules of each clonal line were transferred to half strength hormone-free MS medium supplemented with 100 μM A2C. RA content, proline content, and morphological abnormalities were determined as described in Example 1. Results for one A2C-tolerant line are shown in Table 3.

Ornithine is a precursor of proline in plants. Using oregano clonal line 0-1, the effect of proline, ornithine, and azetidine-2-carboxylate on RA, and proline levels was tested. Culturing line 0-1 on media supplemented with proline, alone or in combination with A2C, resulted in increased levels of proline in shoots at day 30 compared to the untreated control (Table 3). Levels of RA were also increased in these same samples.

When line 0-1 was cultured in the presence of 50 μM A2C, there was no increase in the level of proline in shoots, but there was a significant increase in the amount of RA. These results indicate that shoot tissue cultured for 30 days in the presence of the indicated compounds or combination of compounds results in elevated levels of RA on a fresh weight basis.

TABLE 3
RA and Proline Content in Oregano Line 0-1. Cultured on Media
Supplemented with Proline, Ornithine or A2C

| | RA | Proline | |
|---|---|---|---|
| Media | (mg/g FW ± SD) | (μmol/g FW ± SD). | |
| Treatment | 30d | 15d | 30d |
| Control | 0.89 ± 0.11 | 0.95 ± 0.25 | 0.38 ± 0.22 |
| Proline (4 mM) | 3.51 ± 0.34 | 62.15 ± 9.84 | 18.34 ± 7.40 |
| A2C (50 μM) | 4.28 ± 1.18 | 0.91 ± 0.33 | 0.41 ± 0.17 |
| Ornithine (5 mM) | 4.54 ± 0.92 | 13.33 ± 8.10 | 1.77 ± 0.35 |
| Proline + A2C | 5.06 ± 0.56 | 79.81 ± 10.00 | 5.83 ± 3.49 |
| Ornithine + A2C | 4.09 ± 0.56 | 18.96 ± 2.73 | 0.77 ± 0.33 |

Each value is the mean of 4 separate extracts.

Shoot tissue from oregano lines 0-4 and 0-5 was also cultured in the presence of A2C for 30 days. Samples of treated and control shoots were then assayed for RA content on a fresh weight basis and for morphological abnormalities.

As shown in Table 4, clonal line 0-4 showed no morphological abnormalities after being cultured in the presence of 100 μM azetidine-2-carboxylic acid. In contrast, clonal line 0-5 turned necrotic within about 5 to 7 days of A2C treatment. The RA content of A2C-treated samples of clonal line 0-4 was 4.3 mg/g FW, compared to 2.1 mg/g FW for untreated control samples on day 30. The RA content of clonal line 0-5 was 2.9 mg/g FW for the untreated control samples. Those tissue portions of A2C-treated clonal line 0-5 that were not non-necrotic (primarily shoot tips) were also assayed and had an RA content of 3.2 mg RA/g FW.

TABLE 4
RA Content in Oregano Lines 0-4 and 0-5

| | Media | RA |
|---|---|---|
| Line | Supplement | (mg/g FW ± SD) |
| 0-4 | Control | 2.1 ± 0.4 |
| | A2C | 4.3 ± 0.7 |
| 0-5 | Control | 2.9 ± 0.3 |
| | A2C | 3.2 ± 0.4 |

### Example 3

### Proline Analog Tolerance of High and Low RA-Producing Clonal Lines of Oregano

Oregano lines 0-1, 0-4, and 0-5 of Examples 1 and 2 were tested again for cross-tolerance to A2C. Clonal lines OM-1, and OM-8 of Example 1 were also tested for cross-tolerance to A2C. Line OM-8 was previously shown to be sensitive to Pseudomonas strain F, whereas line OM-1 was tolerant of Pseudomonas strain F. Lines were maintained on MS medium containing BAP as described in Example 1.

Individual shoot tissue propagules of each clonal line were transferred to half strength hormone-free MS medium supplement with A2C. RA content and morphological abnormalities were determined as described in Example 1. The results are shown in Table 5.

Lines 0-5 and OM-8, which are sensitive to-strain F, showed little or no increase in RA content after 30 days of culture in the presence of 100 μM A2C. Lines 0-5 and OM-8 were killed by culturing in the presence of 200 μM A2C.

In contrast, lines 0-1, OM-1, and 0-4, which are tolerant of strain F, had significant increases in RA content. In particular, line OM-1 had a large increase in RA content, and was very tolerant of strain F.

TABLE 5
RA Content in Oregano Cloned Lines Cultured on Media
Supplemented with A2C

| | A-2-C Levels | RA Levels |
|---|---|---|
| Clonal Lines | (μM) | (mg/g FW) ± SD |
| 0-5 | 0 | 2.4 ± 0.3 |
| | 100 | 3.1 ± 0.5 |
| | 200 | inhibited |
| OM-8 | 0 | 2.7 ± 0.2 |
| | 100 | 2.9 ± 0.3 |
| | 200 | inhibited |
| 0-1 | 0 | 2.4 ± 0.7 |
| | 100 | 5.1 ± 0.4 |
| | 200 | 5.4 ± 0.4 |
| OM-1 | 0 | 3.4 ± 0.5 |
| | 100 | 7.1 ± 0.7 |
| | 200 | 6.4 ± 1.2 |
| 0-4 | 0 | 3.4 ± 0.3 |
| | 100 | 6.4 ± 0.8 |
| | 200 | 6.8 ± 0.7 |

### Example 4

### Proline Analog Tolerance of Clonal Lines of Thyme and Rosemary

Three clonal thyme lines and five rosemary clonal lines were isolated and maintained in culture as described in U.S. application Ser. No. 08/771,241, filed Dec. 20, 1996 and incorporated herein by reference.

Individual shoot propagules of each clonal line were transferred to half-strength hormone-free MS media supplemented with A2C and cultured for 30 to 45 days. RA content and morphological abnormalities were determined at the end of the culture period as described in Example 1. Results are shown in Table 6.

Thyme and rosemary clonal lines M-3 and R-1 showed little or no increase in RA content after 30 to 45 days in the presence of 100 μM A2C. Both lines were killed after culture in the presence of 200 μM A2C. Both lines are sensitive to strain F.

The remaining thyme and rosemary lines were tolerant of strain F. All were found to be tolerant of culture in the presence of either 100 or 200 μM A2C. All tolerant lines had significant increases in RA content (Table 6). Lines R-15 and R-16 are useful even though growth is inhibited at 200 μM A2C because these lines have large increases in RA content at 100 μM A2C.

TABLE 6
RA Content in Thyme and Rosemary Clonal Lines
Cultured on Media Supplemented with A2C

| | | A-2-C Levels | RA Levels |
|---|---|---|---|
| Species | Lines | (μM) | (mg/g FW ± SD) |
| Thyme | M-3 | 0 | 1.3 ± 0.4 |
| | | 100 | 2.4 ± 0.5 |
| | | 200 | inhibited |
| | T-16G | 0 | 1.9 ± 0.2 |
| | | 100 | 3.1 ± 0.4 |
| | | 200 | 3.7 ± 0.4 |
| | T-12 | 0 | 2.1 ± 0.4 |
| | | 100 | 4.1 ± 0.5 |
| | | 200 | 5.1 ± 0.5 |
| Rosemary | R-1 | 0 | 3.4 ± 0.2 |
| | | 100 | 4.9 ± 0.5 |
| | | 200 | inhibited |
| | R-7 | 0 | 4.8 ± 0.5 |
| | | 100 | 6.8 ± 1.1 |
| | | 200 | 7.9 ± 2.0 |
| | R-15 | 0 | 5.1 ± 0.7 |
| | | 100 | 9.4 ± 0.6 |
| | | 200 | inhibited |
| | R-16 | 0 | 5.6 ± 0.4 |
| | | 100 | 10.4 ± 1.9 |
| | | 200 | inhibited |
| | R-35 | 0 | 4.7 ± 0.4 |
| | | 100 | 12.1 ± 1.4 |
| | | 200 | 10.9 ± 0.9 |

### Example 5

### Enzyme Assays

Extracts of shoots of oregano line 0-1 were assayed for Glucose-6-phosphate dehydrogenase (G-6-PDH) and phenylalanine ammonia lyase (PAL) activity after 10 and 25 days of culture in the presence of A2C. These enzymes are involved in synthesis of phenolic secondary metabolites (PAL) and in the pentose phosphate pathway (G-6-PDH).

One gm fresh weight of leaves were ground to a powder in liquid nitrogen, and homogenized in 0.1 M Tris-HCl buffer (pH 7.4) containing 1 mM phenylmethyl-sulfonyl fluoride (PMSF), 0.1% polyvinylpyrrolidone (PVP, to remove phenolics and polysaccharides) and 1 mM dithiothreitol (DTT). Extracts were centrifuged at 15,000×g for 20 minutes, and supernatants (5 ml) were partially purified through a PD 10 column (Pharmacia, Inc., NJ). Protein was eluted from the column using 5 ml of 0.1 M Tris-HCl, pH 8.5, which is close to the pH optimum of plant glucose-6-phosphate dehydrogenase (pH 8.0-8.2) and PAL (pH 8.8).

Glucose-6-phosphate dehydrogenase activity in the protein extract was measured spectrophotometrically by following the change in absorbance at 340 nm in the presence of saturating amounts of glucose-6-phosphate (2mM) and NADP (5 μM) in 0.1 M Tris-HCl, pH 8.0. One unit of glucose-6-phosphate dehydrogenase was defined as the amount of enzyme causing the reduction of 1 μmol of NADP⁺ per minute. Specific activity was expressed as units per milligram of protein.

Phenylalanine ammonia-lyase (PAL) activity in the protein extract was assayed based on the conversion of L-phenylalanine (6.67 mM) to trans cinnamic acid. The reaction was carried out at 30° C. in borate buffer (33 mM), pH 8.8 with L-phenylalanine as substrate and the change in absorbance at 290 nm was measured. PAL was expressed as nanomoles of trans-cinnamic acid formed per minute per milligram of protein. Protein was determined by the Bradford dye-binding method using bovine serum albumin as a standard.

The results are shown in Table 7. The activities of glucose-6-phosphate dehydrogenase and PAL were higher at day 10 and day 25 in A2C-treated tissue compared to tissue cultured in the absence of A2C. An increase in enzyme activity is consistent with the hypothesis proposed herein.

TABLE 7
Specific activities of glucose-6-phosphate
dehydrogenase (G-6-PDH) and phenylalanine
ammonia-lyase (PAL) in oregano line 0-1

| | G-6-PDH | | PAL | |
|---|---|---|---|---|
| Treatment | 10d* | 25d | 10d | 25d |
| Control | 0.42 | 0.45 | 0.84 | 0.74 |
| A2C (50 μM) | 1.47 | 1.23 | 2.83 | 2.64 |

G-6-PDE: Specific activity = μmoles NADPH/min/mg protein
PAL: Specific activity = nanomoles cinnamate/min/mg protein
*d-denotes days of culture treatment

### Example 6

### Polymerase Chain Reaction for Identification of Clonal Lines

Two polymerase chain reaction (PCR) methods were used to confirm that clonal lines of oregano were genetically different from each other.

The first method used an Operon 10-mer kit (Operon Tech; CA), which contains random sequence oligonucleotide primers 10 nucleotides in length. A 10-mer (OP10-10) primer with the sequence: 5′GGTCTACACC 3′ [SEQ. ID. NO:3] was used to amplify oregano total DNA. PCR amplification was carried out in a total volume of 25 μl using standard reagents and 1 μl (2 nanograms) of template DNA following the manufacturer's instructions. The amplification conditions were: 94° C. for 1 minute to denature, 50° C. for 1 minute for annealing of primer, and 72° C. for 2 minutes for primer extension, for 45 cycles. PCR amplified products were separated on an agarose gel and product bands were visualized after staining with ethidium bromide. The results showed that M-series oregano clonal lines from a Kentucky gene pool (Advanced Seed Co. Fulton, Ky.) were distinguishable from 0-series clonal lines from a Connecticut gene pool (C.S. Hart Co.).

In the second method, a pair of consensus tRNA gene primers facing outward from tRNA genes were used to amplify oregano total DNA (Welsh and McClelland, 1991). The PCR fingerprints developed from these primers are mainly derived from regions between closely linked tRNA genes. The two primers used were: P#1: 5′AGTCCGTGCTCTAACCAAC 3′ [SEQ. ID. NO: 1]; P#2: 5′GGGGGTTCGAATTCCCGCCGGC 3′ [SEQ. ID. NO:2]. Total oregano DNA was used as a template and amplified as described above. All clonal lines of oregano were clearly different from each other based on the products amplified by the above primers.

### Example 7

### Selection for A2C Tolerance

About 100 oregano clonal lines are initiated as described in Example 1. Lines are maintained on MS medium containing BAP as described in Example 1. At the end of a subculture cycle, 50 shoot propagules of each clonal line are transferred to half-strength hormone-free MS medium containing 200 μM A2C. Propagules are then cultured for 30 days as described in Example 1, and assayed for RA content as described in Example 1. Total phenolic content is measured as described in U.S. application Ser. No. 08/771,241, filed Dec. 20, 1996, incorporated herein by reference.

A small fraction of the lines are found to have an increase in RA and total phenolic content on a fresh weight basis of at least 2-fold, compared to corresponding tissue cultured in the absence of A2C. Growth of these lines is not inhibited by A2C. About half of the lines do not survive culture in the presence of A2C. The remainder of the lines survive the culture period in A2C, and show varying degrees of increase in RA content between 1, and 2-fold.

### Example 8

### Effect of Treatment in a Field Environment

About 100 regenerated plants of oregano clone 0-1, and the five tolerant clones of Example 7 are removed from a greenhouse, and planted outdoors in a field during the spring, and summer growing season. Plantings are carried out using a randomized block design. Plants are fertilized weekly, but are not irrigated. About 7 days before flower emergence, half of the plants of each line are sprayed with an aqueous solution comprising A2C, Nonidet P40™, and EMC prepared from strain F. A2C is present at about 50 μm, and EMC is present at about 0.5% w/v. Each plant receives about 1 to 2 ml on the upper surface of the leaves. The remaining control plants are treated by spraying an aqueous solution that contains no A2C and no EMC. Spraying is carried out such that there is little or no carryover to adjacent plants.

After 7 days, leaf tissue from A2C-treated and control plants is harvested, and analyzed for total phenolic content, and RA content as described in Example 7. The results show that A2C-treated plant tissue has at least 2-fold more total phenolics and at least 2-fold more RA than control tissue on a fresh weight, and on a dry weight basis. The plant-to-plant variation among A2C-treated plants of each line is about 5% to about 15%.

### Other Embodiments

It is to be understood that while the invention has been described in conjunction with the Detailed Description thereof, that the foregoing description is intended to illustrate, and not limit the scope of the invention, which is defined by the scope of the appended claims. Other aspects, advantages, and modifications are within the scope of the following claims.

What is claimed is:

1. A method for producing a clonal plant line containing an elevated level of a secondary metabolite, said method comprising the steps of:
a) culturing a first propagule of a Lamiaceae clonal line in the presence of a compound that stimulates proline metabolic flux; and
b) comparing the level of said secondary metabolite in said first propagule to the level of said secondary metabolite in a second propagule of said clonal line cultured in the absence of said compound; and
c) using said comparison to determine whether said propagule has an elevated level of said secondary metabolite relative to said level in said second propagule, and if so, using said first propagule to produce the clonal plant line.

2. The method of claim 1, wherein said clonal line is from the species Mentha spicata.

3. The method of claim 1, wherein said clonal line is from the species Thymus vulgaris L.

4. The method of claim 1, wherein said clonal line is from the species Origanum vulgare.

5. The method of claim 1, wherein said clonal line is from the species Rosmarinus officinalis.

6. The method of claim 1, wherein said clonal line is from the species Melissa officinalis.

7. The method of claim 1, wherein said clonal line is from the species Lavandula augustifolia.

8. The method of claim 2, wherein said secondary metabolite is rosmarinic acid.

9. The method of claim 3, wherein said secondary metabolite is thymol or carvacrol.

10. The method of claim 4, wherein said secondary metabolite is rosmarinic acid.

11. The method of claim 5, wherein said secondary metabolite is rosmarinic acid.

12. The method of claim 6, wherein said secondary metabolite is rosmarinic acid.

13. The method of claim 7, wherein said secondary metabolite is rosmarinic acid.

14. The method of claim 1, further comprising the step of regenerating at least one plant from said first propagule.

15. The method of claim 1, wherein said proline-flux stimulating compound is a competitive inhibitor of proline dehydrogenase.

16. The method of claim 15, wherein said compound is azetidine-2-carboxylic acid.

17. The method of claim 16, wherein said compound is present at about 50 μM to about 350 μM.

18. A Lamiaceae plant produced by:
a) culturing a propagule of a Lamiaceae clonal line in the presence of a compound that stimulates proline metabolic flux; and
b) regenerating said plant from said propagule, said propagule surviving said culturing.

19. The plant of claim 18, wherein said plant is Mentha spicata.

20. The plant of claim 18, wherein said plant is Thymus vulgaris L.

21. The plant of claim 18, wherein said plant is Origanum vulgare.

22. The plant of claim 18, wherein said plant is Rosmarinus officinalis.

23. The plant of claim 18, wherein said species is Melissa officinalis.

24. The plant of claim 18, wherein said species is Lavandula augustifolia.

25. The plant of claim 18, wherein said proline-flux stimulating compound is a competitive inhibitor of proline dehydrogenase.

26. The plant of claim 25, wherein said compound is azetidine-2-carboxylic acid.

27. The plant of claim 26, wherein said compound is present at about 50 μM to about 350 μM.

28. The plant of claim 18, wherein the level of a secondary metabolite in said cultured propagule is compared to the level of said secondary metabolite in a control propagule of said clonal line cultured in the absence of said compound, said cultured propagule having an elevated level of said secondary metabolite relative to said level in said control propagule.

29. The plant of claim 28, wherein said plant has an elevated level of said secondary metabolite relative to said level in a plant regenerated from said control propagule.

30. A method for producing a plant containing an elevated level of a secondary metabolite, said method comprising the steps of:
a) culturing propagules from a plurality of Lamiaceae clonal lines in the presence of a proline flux-stimulating compound;
b) selecting propagules from at least one line that is tolerant of said compound, said selected propagules containing an elevated level of said secondary metabolite relative to the corresponding secondary metabolite level in control propagules of said selected line cultured in the absence of said compound; and
c) regenerating at least one plant from said selected propagules.

31. The method of claim 30, wherein compound is a competitive inhibitor of proline dehydrogenase.

32. The method of claim 31, wherein said compound is azetidine-2-carboxylic acid.

33. The method of claim 32, wherein said compound is present at about 50 μM to about 350 μM.

34. A method for producing an elevated level of a secondary metabolite in a Lamiaceae plant, said method comprising the steps of:
a) obtaining a Lamiaceae plant selected for tolerance to a proline flux-stimulating compound;
b) contacting said plant with said compound; and
c) growing said plant for a time sufficient to produce an elevated level of said secondary metabolite.

35. The method of claim 34, wherein said compound is hydroxyproline.
